(19) United States
(12) Patent Application Publication
(10) Pub. No.: US 2016/0287228 A1
(43) Pub. Date: Oct. 6, 2016

(54) AMNIO OPENING OCCLUSION DEVICE

(71) Applicant: Ruben Quintero, Miami, FL (US)

(72) Inventor: Ruben Quintero, Miami, FL (US)

(21) Appl. No.: 15/087,719

(22) Filed: Mar. 31, 2016

Related U.S. Application Data
(60) Provisional application No. 62/140,879, filed on Mar. 31, 2015.

(51) Int. Cl. A61B 17/00; A61B 17/12

(52) U.S. Cl. CPC A61B 17/0057; A61B 17/12031; A61B 17/12099; A61B 17/12172; A61B 2017/00606; A61B 2017/00867

(57) ABSTRACT

The disclosed collapsible medical device is used for occluding an opening used for fetoscopic surgery. The device comprises a tubular woven metal fabric comprised of a plurality of braided metal strands and having a proximal end and a distal end, and said fabric having an expanded preset configuration comprising two disk-shaped elements and a waist element; wherein said two disk-shaped elements have diameters of about 5 mm; wherein the waist element has a length of about one centimeter; wherein the fabric is deformable to a lesser cross-sectional dimension for delivery through a channel in a patient's body to said opening used for fetoscopic surgery, and the fabric having a memory property whereby the medical device tends to return to said expanded preset configuration when unconstrained.

### CROSS-REFERENCE TO RELATED APPLICATIONS

This application claims priority to provisional application No. 62/140,879 filed Mar. 31, 2015 and entitled AMNIO OPENING OCCLUSION DEVICE. The subject matter of application No. 62/140,879 is hereby incorporated by reference in its entirety.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The disclosed embodiments relate to the field of medical devices and more specifically to medical devices used to close or occlude an opening in a patient.

### BACKGROUND

Fetal surgery is any of a broad range of surgical techniques and processes that are used to treat birth defects or other medical problems in fetuses who are still in the pregnant uterus. Open fetal surgery involves completely opening the uterus to operate on the fetus. Minimally invasive fetoscopic surgery (fetendo) uses small incisions and is guided by fetoscopy and sonography. Minimally-invasive fetoscopic surgery uses real-time video imagery from fetoscopy and ultrasonography to guide very small surgical instruments into the uterus in order to surgically help the fetus. Less invasive than open fetal surgery, some fetal surgeries can be achieved with just a small incision on the skin of the mother (percutaneous), though in some cases it may require that a small opening be made in the mother's abdomen. The fact that it is less invasive reduces the mother's postoperative recovery and lessens the troubles with preterm labor. Minimally-invasive fetoscopic surgery has proven to be very useful for most, but not all, fetal or placental conditions

One of the problems associated with even minimally-invasive fetoscopic surgery is the opening that is made in the pregnant patient's uterus in order to operate on the fetus. This opening, which results from the insertion of the trocar or other medical instruments, can be the cause of later problems in the patient. Said opening can cause the fetal membrane to separate from the uterine wall, otherwise known as chorioamniotic separation or membrane detachment, which can lead to complications. Chorioamniotic separation may result either in extravasation of the amniotic fluid between the amnion and the chorion, which may hinder further access to the amniotic cavity, or in gross leakage of fluid via the patient's vagina. In the latter case, the pregnancy is at risk for being lost due to premature rupture of said membranes. Said leakage through the opening and out of the vagina can further alarm the patient and result in infection or other difficulties.

Therefore, there exists a need for improvements over the prior art, and more particularly for a more efficient way of treating the opening that is made in the pregnant patient's body during fetoscopic surgery.

### SUMMARY

In one embodiment, a medical device is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

The disclosed collapsible medical device is used for occluding an opening used for fetoscopic surgery. The device comprises a tubular woven metal fabric comprised of a plurality of braided metal strands and having a proximal end and a distal end. said woven metal fabric having an expanded preset configuration comprising two disk-shaped elements and a waist element in between the two disk-shaped elements; wherein said two disk-shaped elements have diameters of about 5 mm; wherein the waist element has a length of about one centimeter and a diameter that is less than a diameter of the two disk-shaped elements; wherein the woven metal fabric is deformable to a lesser cross-sectional dimension for delivery through a channel in a patient's body to said opening used for fetoscopic surgery, and the woven metal fabric having a memory property whereby the medical device tends to return to said expanded preset configuration when unconstrained.

### BRIEF DESCRIPTION OF THE DRAWINGS

The accompanying drawings, which are incorporated in and constitute part of this specification, illustrate embodiments of the claimed subject matter and together with the description, serve to explain the principles of the invention. The embodiments illustrated herein are presently preferred, it being understood, however, that the invention is not limited to the precise arrangements and instrumentalities shown, wherein:

FIG. 1 is a side view of the collapsible medical device in a semi-deployed mode, according to one embodiment.

FIG. 2 is a side view of the collapsible medical device in a semi-deployed mode, according to one embodiment.

FIG. 3 is a side view of the collapsible medical device in a deployed mode, according to one embodiment.

FIG. 4 is a side view of the collapsible medical device in a deployed mode and used in an opening used for fetoscopic surgery, according to one embodiment.

### DETAILED DESCRIPTION

The disclosed embodiments improve upon the problems with the prior art by providing a collapsible medical device that is easily deployed for use in occluding an opening used for fetoscopic surgery. The disclosed embodiments improve over the prior art by providing a safe, inexpensive and user-friendly method for occluding an opening used for fetoscopic surgery. Additionally, the disclosed embodiments improve over the prior art by providing a medical device that prevents or reduces the incidence of chorioamniotic separation or membrane detachment, which can otherwise lead to complications. Further, the disclosed embodiments reduce the likelihood of losing a pregnancy due to premature rupture of said membranes, as well as the likelihood of infection and other maternal and fetal difficulties. Further, the disclosed embodiments improve over the prior art by providing a medical device that is easily deployable via a catheter or trocar, thereby reducing the number or size of incisions that must be made in a patient's body.

The disclosed embodiments are formed from a resilient metal fabric a plurality of resilient strands or wires, with the metal fabric being formed by braiding the resilient strands to create a resilient material. This braided fabric is then deformed to generally conform to a molding surface of a molding element and the braided fabric is heat treated in contact with the surface of the molding element at an elevated temperature. The time and temperature of the heat treatment is selected to substantially set the braided fabric in its deformed state. After the heat treatment, the fabric is removed from contact with the molding element and will substantially retain its shape in the deformed state. The braided fabric so treated defines a relaxed state of a medical device which can be stretched or expanded and deployed through a catheter into a channel in a patient's body.

The device of the present invention has a specific shape which is particularly well suited for occluding an opening made in a uterus during fetendo. The device has a relaxed low-profile configuration and includes clamps that allow attachment of the device to an end of a delivery device or guide wire (allowing recovery of the device after placement). In use, a guide catheter is positioned and advanced in a patient's body such that the distal end of the catheter is adjacent a desired treatment site for treating a physiological condition. The medical device of the present invention having a predetermined shape is then stretched and inserted into the lumen of the catheter. The device is urged through the catheter and out the distal end, whereupon, due to its shape memory property it will tend to substantially return to its relaxed state adjacent the treatment site. The guide wire or delivery catheter is then released from the clamp and removed.

The collapsible occluding device includes two spaced apart occluding members (shaped like disks) interconnected by a flexible, resilient center portion (called a waist). In the preferred embodiment, the occluding device is formed from a single continuous tubular metal fabric. The tubular fabric is formed from a plurality of wire strands having a predetermined relative orientation between the strands. This tubular fabric is known in the fabric industry as a tubular braid.

Once a device having a preselected shape has been formed, the device may be used to treat a physiological condition of a patient. A medical device suitable for treating the condition is selected. Once the appropriate medical device is selected, a catheter or other suitable delivery device may be positioned within a channel in a patient's body to place the distal end of the delivery device adjacent the desired treatment cite, such as immediately adjacent (or even within) the shunt of an abnormal opening in the patient's organ for example.

The delivery device (not shown) can take any suitable shape, but desirably comprises an elongate flexible metal shaft having a threaded distal end. The delivery device can be used to urge the medical device through the lumen of a catheter for deployment in a channel of a patient's body. When the device is deployed out the distal end of the catheter, the device will still be retained by the delivery device. Once the medical device is properly positioned within the shunt of the abnormal opening, the distal end of the catheter may be pressed against the medical device and the metal shaft or guidewire can be rotated about its axis to unscrew the medical device from the threaded distal end of the shaft. The catheter and guidewire are then withdrawn.

By keeping the medical device attached to the delivery means, the operator can retract the device for repositioning relative to the abnormal opening, if it is determined that the device is not properly positioned within the shunt. A threaded clamp attached to the medical device allows the operator to control the manner in which the medical device is deployed out the distal end of the catheter. When the device exits the catheter, it will tend to resiliently return to a preferred relaxed shape. When the device springs back into this shape, it may tend to act against the distal end of the catheter effectively urging itself forward beyond the end of the catheter. This spring action could conceivably result in improper positioning of the device if the location of the device within a channel is critical, such as where it is being positioned in a shunt between two vessels. Since the threaded clamp can enable the operator to maintain a hold on the device during deployment, the spring action of the device can be controlled by the operator to ensure proper positioning during deployment.

The medical device can be collapsed into its collapsed configuration and inserted into the lumen of the catheter. The collapsed configuration of the device may be of any shape suitable for easy passage through the lumen of a catheter and proper deployment out the distal end of the catheter. For example, the device may have a relatively elongated collapsed configuration wherein the device is stretched along its longitudinal axis (see FIG. 1). This collapsed configuration can be achieved simply by stretching the device generally along its axis, e.g. by manually grasping the clamps and pulling them apart, which will tend to collapse the relaxed diameter portions of the device inwardly toward the device's axis. Loading such a device into a catheter may be done at the time of implantation and does not require pre-loading of the introducer or catheter.

If the device is to be used to permanently occlude a channel in the patient's body, one can simply retract the catheter and remove it from the patient's body. This leaves the medical device deployed in the patient's vascular system so that it may occlude the blood vessel or other channel in the patient's body. In some circumstances, the medical device may be attached to a delivery system in such a manner as to secure the device to the end of the delivery means. Before removing the catheter in such a system, it may be necessary to detach the medical device from the delivery means before removing the catheter and the delivery means.

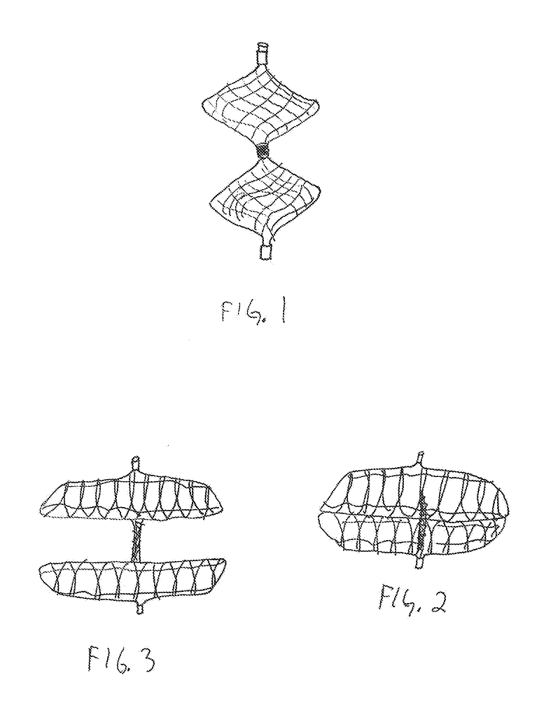

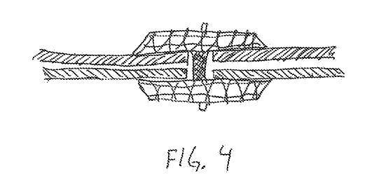

Having described the details of the invention, specific reference to the Figures will next be presented. The several Figures illustrate several embodiments of the invention wherein the outer discs pull towards each other. Referring first to the FIGS. 1-4, there is shown generally the device suitable for occluding an opening in a uterus made during fetendo. In its relaxed, unstretched state (see FIGS. 3-4), the device generally includes two aligned discs and linked together by a resilient central portion or waist. The plurality of braided wires form an outer and inner surface of each disc. The inner surface of each disc may be concave or cupped to ensure that the outer perimeter edge of each disc respective contacts the wall of the opening being occluded.

When the device is in a relaxed state, the discs and tend to overlap and the central portion extends into the recess formed by the inner surface of the discs. In this manner, when the discs are pulled apart (see FIGS. 1 and 3) the spring-like action of the central portion will cause the perimeter edge of the corresponding disc to fully engage the sidewall of the occlusion (see FIG. 4). FIGS. 1-3 illustrate sequentially the stretching, spring-like action of the device.

Those skilled in the art will appreciate that the device is sized in proportion to the opening to be occluded. The diameter of each disc may be varied as desired for differently sized openings in the uterus. If the size of the opening is 3.8 mm, then the size of the discs is 5 mm; if the size of the opening is 5 mm, then the size of the discs is 7 mm; if the size of the opening is 7 mm, then the size of the discs is 9 mm Both discs may be the same size. Further, the length of the resilient central portion—the waist—may be varied depending upon the thickness of the uterine wall and the fetal membrane, and may range between 0.5 to 1 cm.

The well-known Amplatzer device should be addressed here. The present invention is based on its use in a part of the body that is different from the part of the body for which the Amplatzer device was originally intended. The Amplatzer device is designed to generate cell ingrowth in the heart to seal off atrial septal defects. When placed in the uterus, the Applicant's device does not generate cell ingrowth and can be easily removed at the time of delivery of the baby. This represents an unexpected reaction/result from the use of the Amplatzer device in the human body.

Although specific embodiments have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the invention.

We claim:

1. A collapsible medical device for occluding an opening used for fetoscopic surgery, comprising:
a tubular woven metal fabric comprised of a plurality of braided metal strands and having a proximal end and a distal end
said woven metal fabric having an expanded preset configuration comprising two disk-shaped elements and a waist element in between the two disk-shaped elements;
wherein said two disk-shaped elements have diameters of about 5 mm;
wherein the waist element has a length of about one centimeter and a diameter that is less than a diameter of the two disk-shaped elements;
wherein the woven metal fabric is deformable to a lesser cross-sectional dimension for delivery through a channel in a patient's body to said opening used for fetoscopic surgery; and
the woven metal fabric having a memory property whereby the medical device tends to return to said expanded preset configuration when unconstrained.

2. A collapsible medical device for occluding an opening used for fetoscopic surgery, comprising:
a tubular woven metal fabric comprised of a plurality of braided metal strands and having a proximal end and a distal end
said woven metal fabric having an expanded preset configuration comprising two disk-shaped elements and a waist element in between the two disk-shaped elements;
wherein said two disk-shaped elements have diameters of about 7 mm;
wherein the waist element has a length of about one centimeter and a diameter that is less than a diameter of the two disk-shaped elements;
wherein the woven metal fabric is deformable to a lesser cross-sectional dimension for delivery through a channel in a patient's body to said opening used for fetoscopic surgery; and
the woven metal fabric having a memory property whereby the medical device tends to return to said expanded preset configuration when unconstrained.

3. A collapsible medical device for occluding an opening used for fetoscopic surgery, comprising:
a tubular woven metal fabric comprised of a plurality of braided metal strands and having a proximal end and a distal end
said woven metal fabric having an expanded preset configuration comprising two disk-shaped elements and a waist element in between the two disk-shaped elements;
wherein said two disk-shaped elements have diameters of about 9 mm;
wherein the waist element has a length of about one centimeter and a diameter that is less than a diameter of the two disk-shaped elements;
wherein the woven metal fabric is deformable to a lesser cross-sectional dimension for delivery through a channel in a patient's body to said opening used for fetoscopic surgery; and
the woven metal fabric having a memory property whereby the medical device tends to return to said expanded preset configuration when unconstrained.
